(19) United States
(12) Patent Application Publication
(10) Pub. No.: US 2010/0068232 A1
(43) Pub. Date: Mar. 18, 2010

(54) Methods for treating skin lines and wrinkles and improving skin quality

(76) Inventor: Douglas J. Key, Portland, OR (US)

Correspondence Address: KLARQUIST SPARKMAN, LLP, 121 SW SALMON STREET, SUITE 1600, PORTLAND, OR, 97204, US

(21) Appl. No.: 12/583,944

(22) Filed: Aug. 27, 2009

Related U.S. Application Data
(60) Provisional application No. 61/190,562, filed on Aug. 28, 2008.

(51) Int. Cl. A61K 39/08; A61P 21/00

(52) U.S. Cl. 424/239.1

(57) ABSTRACT

Methods and compositions for improving skin quality are disclosed. The methods include applying a solution of about 20-30% pre-wetting agent, such as hyaluronic acid, and/or Botulinum toxin to a skin surface with concurrent particle-free dermabrasion treatment, such as particle-free microdermabrasion treatment. These methods lead to significant improvement in the treatment of skin aging. Compositions for improving skin quality are also disclosed. These compositions comprise a pre-wetting agent and a Botulinum toxin agent.

### CROSS REFERENCE TO RELATED APPLICATION

This application claims the benefit of U.S. Provisional Patent Application No. 61/190,562, filed Aug. 28, 2008, which is incorporated herein by reference in its entirety.

### FIELD

This disclosure relates to the field of skin treatment, more specifically to the use of needle-free penetration of physiologic or pharmacologic agents such as hyaluronic acid, chondroitin sulfate, or collagen, and/or Botulinum toxin, by a method of wetted, particle-free dermabrasion to improve the appearance of the skin.

### BACKGROUND

Many methods are used to improve skin quality, reduce age spots, soften fine lines, and treat acne or other scars, including dermabrasion, chemical peeling, laser resurfacing, and microdermabrasion. Microdermabrasion removes dead cells from the outermost layer of the skin, referred to as the epidermis, cleans out blocked pores, and enhances skin tone. In this technique, generally the skin is abraded with a high-pressure flow of microcrystals, typically quartz, metal, or aluminum oxide. Chemicals, ultrasonic oscillating tips and lasers have also been used for a more aggressive abrasion (see, for example, U.S. Pat. No. 5,012,797).

Hyaluronic acid is found in all tissues in the body, where it functions to retain water, and provide nutrients and remove waste from cells that do not have a direct blood supply (Block and Bettelheim (1970) Biochim. Biophys. Acta 201:69). It is the matrix within which both collagen and elastic tissue fibers are imbedded. It has been used to treat osteoarthritis, by injection directly into the synovial fluid in the knee (George (1998) Ann. Rheum. Dis. 57:637-40; Weiss et al. (1981) Arthritis Rheum. 11 (suppl 1):143-4). Hyaluronic acid injection into the skin has been used to improve the skin's contour and reduce depressions in the skin due to acne, scars, injury or lines (Duranti et al. (1998) Dermatol. Surg. 24:1317-25; Jordan (2003) Can. J. Opthalmol. 38:285-8; Cheng et al. (2002) Otolaryngol. Clin. North Am. 35:73-85). In addition, hyaluronic acid may be taken orally as a nutritional supplement.

Chondroitin sulfate (CS) and glucosamine are components of the matrix of cartilage. They help hydrate the cartilage by maintaining osmotic pressure through the absorption of water. CS also contributes to the flexibility and elasticity of the bone. It serves as a chondroprotective agent by protecting the cartilage against enzymatic reactions and free radical damage (including the nitric oxide released by chondrocytes). CS is used in treating osteoarthritis and is usually prescribed along with glucosamine sulfate, where the former helps prevent further damage and the latter helps form cartilage.

Collagen is the main protein of connective tissue in animals, the main component of cartilage, ligaments and tendons, and the main protein component of bone and teeth. Along with soft keratin, it is responsible for skin strength and elasticity, and its degradation leads to wrinkles that accompany aging.

Botulinum toxin (BTX), produced by the bacterium Clostridium botulinum reversibly paralyzes striated muscle when administered in sub-lethal doses. BTX has been used in the treatment in a number of neuromuscular disorders and conditions involving muscular spasm including, but not limited to, dystonia, hemifacial spasm, tremor, spasticity (e.g., resulting from multiple sclerosis), anal fissures and various ophthalmologic conditions (for example, see Carruthers et al., J. Amer. Acad. Derm. 34:788-797, 1996). BTX has also been used for cosmetic and subjective needs, for example, altering or improving the appearance of facial tissue. One cosmetic form of BTX, often referred to by its product name BOTOX®, is a popular non-surgical injection that temporarily reduces or eliminates frown lines, forehead creases, crows feet near the eyes and thick bands in the neck. The clinical effects of peripheral intramuscular BTX type A are usually seen within one week of injection. The typical duration of symptomatic relief from a single intramuscular injection of BTX type A averages about three months.

### SUMMARY

The use of injectable hyaluronic acid or BTX for altering or improving the appearance of facial tissue has become extremely popular, but it can be painful and there are possible side effects including bruising, redness, pain, itching, tenderness and swelling at the site of injection. For example, many subjects find the injections to be painful while others cannot undergo the treatment at all because of a fear of needles. The use of injections also limits the treatment surface area that can be covered and requires a trained professional for administration. Therefore, methods and compositions for improving skin quality without the use of injections and without the risk of swelling and bruising are needed. In addition, methods and compositions for improving skin quality that can be performed or used at home, such as provided in a kit, are desirable.

A method and composition for improving skin quality are disclosed. The method includes applying a solution of at least about 20% to about 30% weight/volume (W/V) of a pre-wetting agent such as hyaluronic acid, chondroitin sulfate, or collagen, and/or BTX to a skin surface in conjunction with a dermabrasion treatment, such as particle-free microdermabrasion. The method can be performed by a clinician or other healthcare provider. The method can reduce the appearance of skin changes associated with aging, visibly reduce human skin wrinkles, and improve the textural quality of skin. Compositions and kits for improving skin quality are provided that include a pre-wetting agent and/or BTX. Compositions and kits for improving skin quality are also provided that include a pre-wetting agent and/or an abrasive agent.

The foregoing and other features and advantages of the disclosure will become more apparent from the following detailed description of a several embodiments which proceed with reference to the accompanying figures.

### BRIEF DESCRIPTION OF THE DRAWINGS

FIG. 1 is a graph showing the periorbit subjective scoring of right saline-wetted microdermabrasion with iontophoresis versus left 30% hyaluronic acid-wetted microdermabrasion with iontophoresis.

FIG. 2 is a graph showing the periorbit subjective percent change of right saline-wetted microdermabrasion with iontophoresis versus left 30% hyaluronic acid-wetted microdermabrasion with iontophoresis.

FIG. 3 is a graph showing the periorbit objective scoring of right saline prewetted microdermabrasion with iontophoresis versus left 30% hyaluronic acid prewetted microdermabrasion with iontophoresis.

FIG. 4 is a graph showing the periorbit objective percent change of right saline-wetted microdermabrasion with iontophoresis versus left 30% hyaluronic acid-wetted microdermabrasion with iontophoresis.

FIG. 5 is a graph showing the upper lip subjective scoring of right saline prewetted microdermabrasion with iontophoresis versus left 30% hyaluronic acid prewetted microdermabrasion with iontophoresis.

FIG. 6 is a graph showing the upper lip subjective percent change of right saline-wetted microdermabrasion with iontophoresis alone versus left 30% hyaluronic acid-wetted microdermabrasion with iontophoresis.

FIG. 7A is a digital image showing 30% hyaluronic acid-wetted particle-free microdermabrasion with iontophoresis to the left periorbit and upper lip and 30% hyaluronic acid-wetted particle-free microdermabrasion to the left cheek but without iontophoresis (right) versus pre-treatment control (left). FIG. 7B is a digital image showing detail inserts of the perioral pericheek aspect.

FIG. 8 is a graph showing cheek subjective score of right saline-wetted microdermabrasion versus left hyaluronic acid-wetted microdermabrasion.

FIG. 9 is a graph showing the cheek subjective percent change of right saline-wetted microdermabrasion versus left 30% hyaluronic acid-wetted microdermabrasion.

FIG. 10 is a graph showing cheek objective score of right saline-wetted microdermabrasion versus left hyaluronic acid-wetted microdermabrasion.

FIG. 11 is a graph showing the cheek objective panel scoring of the right saline-wetted microdermabrasion versus left 30% hyaluronic acid-wetted microdermabrasion.

FIG. 12 is a graph showing the comparison between the left cheek and left periorbit (subjective scores).

FIG. 13 is a graph showing the subjective scores percent change of the left cheek versus left periorbit.

FIG. 14 is a graph showing the comparison between the left cheek versus left perioral region (subjective scores).

FIG. 15 is a graph showing the subjective score percent change of the left cheek versus left perioral.

FIG. 16A is a digital image showing the left 30% hyaluronic acid-wetted microdermabrasion cheek, before (left) and after (right) treatment. FIG. 16B is a digital image showing a detailed view of the eye area, pre- (left) and post- (right) treatment.

FIG. 17 is a graph showing the overall subjective score of the right half face saline-wetted microdermabrasion with iontophoresis (periorbit and upper lip) versus the left half face 30% hyaluronic acid-wetted microdermabrasion with iontophoresis (periorbit and upper lip).

FIG. 18 is a graph showing the percent change of the overall subjective score of the right half face saline-wetted microdermabrasion with iontophoresis (periorbit and upper lip) versus the left half face 30% hyaluronic acid-wetted microdermabrasion with iontophoresis (periorbit and upper lip).

FIG. 19 is a graph showing the overall objective score of the right half face saline-wetted microdermabrasion with iontophoresis (periorbit and upper lip) versus the left half face 30% hyaluronic acid-wetted microdermabrasion with iontophoresis (periorbit and upper lip).

FIG. 20 is a graph showing the overall objective score percent change of the right half-face saline-wetted microdermabrasion with iontophoresis (periorbit and upper lip) versus left half-face 30% hyaluronic acid-wetted microdermabrasion with iontophoresis (periorbit and upper lip).

FIG. 21 is a graph showing the comparison of average scores for the right periorbit HA microdermabrasion versus the left periorbit HA microdermabrasion with iontophoresis

FIG. 22 is a graph showing the comparison of average ratings of applied saline (right) versus applied hyaluronic acid (left) without microdermabrasion treatment.

### DETAILED DESCRIPTION

The surprising finding that improvement of skin quality can be obtained by applying high concentrations of one or more enhancing agents for pre-wetting the skin (pre-wetting agent) in conjunction with dermabrasion (such as microdermabrasion) is disclosed herein. This disclosure is related to U.S. patent application Ser. No. 11/534,174 which claims the benefit of U.S. Provisional Patent Application No. 60/719,917, filed Sep. 22, 2005 and U.S. Provisional Application No. 60/776,520, filed Feb. 24, 2006, each of which is incorporated herein by reference in its entirety.

In some embodiments, the skin can be pre-wetted with a solution or gel of any compound of interest, including but not limited to collagen, chondroitin sulfate, or hyaluronic acid and treated with dermabrasion. In one example, BTX can be applied to the skin surface and then the skin surface can be treated with dermabrasion, such as particle-free dermabrasion. In some examples, the skin is pre-wetted with a pre-wetting agent, such as collagen, chondroitin sulfate, or hyaluronic acid in combination with BTX, and treated with dermabrasion, such as particle-free dermabrasion. By “pre-wetted” it is intended that the skin surface be wet prior and during the dermabrasion treatment, such as a microdermabrasion treatment. In one example, the skin is saturated with a solution of a therapeutic amount of one or more agents immediately prior to or concurrent with dermabrasion (such as microdermabrasion), in which the one or more agents can include BTX and/or a pre-wetting agent. The agent can be in the form of a lotion, gel, cream, aqueous solution, or in an ointment or oil base, or in a sprayable liquid form. In one example, the epidermis is saturated with the pre-wetting agent. In another example, there is penetration of the pre-wetting agent into the dermis.

In one example, a therapeutically effective amount of an agent, such as BTX either alone or in combination with hyaluronic acid, is applied directly to the skin surface, in conjunction with dermabrasion, such as microdermabrasion (e.g., particle-free microdermabrasion). In other examples, chondroitin sulfate or collagen either alone or in combination with BTX are applied directly to the skin surface, in conjunction with dermabrasion, such as microdermabrasion (e.g., particle-free microdermabrasion). This method leads to a dramatic enhancement in the effacement of lines and wrinkles. Methods for improving skin quality are also provided, including a reduction in roughness, dryness, scaliness, fine lines and wrinkles, scarring, age spots, and any improvement in texture. The method can result in an increase in luster and brightness of the skin surface. The methods include applying a composition of at least about 20% weight per volume (W/V), such as a composition of about 20% to about 30% W/V pre-wetting agent either alone or in combination with a sufficient concentration of BTX to a skin surface with dermabrasion treatment to bring about the therapeutic desirable effect (without producing adverse or undesired results). In some examples, the concentration of BTX is at least 10 units of BTX, such as about 20 units to about 200 units of BTX, about 30 to about 100 units, or about 30 units to about 50 units, including 15 units, 20 units, 25 units, 30 units, 40 units, 50 units, 60 units, 70 units, 80 units, 100 units, 120 units, 150 units, 200 units. Unit equivalents is an amount of Botulinum Toxin (BTX) that is equivalent to standard Units of Botulinum Toxin A (BTX-A). A standard Unit of BTX-A is defined as the mean LD₅₀ for female Swiss Webster mice weighing 18-20 grams (Schantz and Kaultner, J. Assoc. Anal. Chem. 61: 96-99, 1978). The estimated human LD₅₀ for a 70-kg person is 40 Units/kg or about 2500-3000 Units.

BOTOX™ (Botulinum toxin A, Allergan, Inc., Irvine, Calif., U.S.A.) is sold in 100 Unit vials. DYSPORT™ (Speywood Pharmaceuticals, Ltd., Maidenhead, U.K.) is sold in 500 Unit vials. For cosmetic uses, the vial contents are typically diluted with 1 or 2 ml of sterile saline solution, which for BOTOX™ provides a 100 or 50 Unit/ml dilution. DYSPORT™ BTX-A is roughly tenfold less toxic than BOTOX™ and approximately fourfold greater amounts of the DYSPORT™ product will usually be injected to achieve the same result as would be obtained using a specific number of Units of BOTOX™.

In other embodiments, the composition includes at least a 30% W/V solution of pre-wetting agent, such as about 30% W/V to about 50% W/V composition of a pre-wetting agent, or about 20% W/V, about 25% W/V, about 30% W/V, or greater than about 25% W/V composition of a pre-wetting agent. As discussed above, the pre-wetting agent can be any compound of interest, including, but not limited to, collagen, chondroitin sulfate, or hyaluronic acid either alone or in combination with BTX, and can be in any form, including any lotion, gel, cream, or in an ointment or oil base, or sprayable liquid form that includes a therapeutically effective amount of the agent and is applied to the skin surface without injection. In one embodiment, the pre-wetting agent is any compound that increases the efficacy of BTX. BTX can be administered via the same composition as the pre-wetting agent, such as the pre-wetting agent can be used to dilute the BTX so that the desired concentration of BTX and the pre-wetting agent are obtained. In other embodiments, BTX and the pre-wetting agent are administered via different compositions, but are applied to a skin surface with dermabrasion treatment, such as particle-free dermabrasion treatment. In some embodiments, only BTX is administered.

By “dermabrasion” is intended a procedure in which the surface of the skin is disrupted by abrasion, generally not deeper than the epidermis. Dermabrasion treatments include microdermabrasion treatments. Thus, when the term “dermabrasion” is used in this disclosure, it encompasses microdermabrasion. It generally involves controlled surgical scraping that ‘refinishes’ the top layers of the skin to give a smoother appearance. By “microdermabrasion” is intended a procedure in which the stratum cornea (the outermost surface of the skin) is removed by light abrasion. By “abrasive agent” is intended an agent capable of being used in a dermabrasion treatment, such as a microdermabrasion treatment.

Any skin surface can be treated using the methods provided herein. By “skin surface” is intended the epidermis of the skin. Skin surfaces that can be treated include, but are not limited to, periorbits, lips, cheeks, nasolabial folds, forehead, neck, upper lip rhytides, neck, back, chest, hands and feet, or any combination thereof. The skin of any facial surface can be treated using the methods provided herein. The method can be applied to any facial area and/or to any body surface area, with other immediate areas of application being the chest and neck. More than one skin surface can be treated during the same treatment period.

In several examples, a composition of about 20% to about 30% solution of pre-wetting agent (W/V) and/or BTX is applied immediately prior to dermabrasion, such as microdermabrasion, such as stratum cornea. In several examples, about 25%, about 26%, about 27%, about 28%, about 29% or about 30% pre-wetting agent (W/V) is utilized, including 25%, 26%, 27%, 28%, 29% or 30% pre-wetting agent. The term “about” includes within 2% of the given percentage of the pre-wetting agent W/V. In some examples, the concentration of BTX is at least 10 units of BTX, such as about 20 units to about 200 units of BTX, about 30 to about 100 units, or about 30 units to about 50 units, including 15 units, 20 units, 25 units, 30 units, 40 units, 50 units, 60 units, 70 units, 80 units, 100 units, 120 units, 150 units, 200 units. The term “about” includes within 2 units equivalents of the given units equivalents. By “in conjunction with” or “concurrent” treatment is intended the application of pre-wetting agent and BTX from about zero to about two hours (such as simultaneously, within 1 minute, within 2 minutes, within 5 minutes, 10 minutes, 30 minutes, 50 minutes, 90 minutes or 120 minutes) prior to dermabrasion (such as microdermabrasion), such that a sufficient amount of the pre-wetting agent and BTX are present at the time of dermabrasion. In one embodiment, “in conjunction with” or “concurrent” treatment is a treatment performed during a single visit to a clinician, for example within a few minutes to a few hours of treatment. In another embodiment, the “concurrent” treatment is performed at home, with the use of a kit that contains a pre-wetting composition (comprising the pre-wetting agent) and/or a BTX composition (comprising BTX), with instructions for use. With respect to the at-home treatment product, the pre-wetting composition and/or the BTX composition can be packaged together in one composition, or can be separate compositions that are applied during the same treatment period. Generally, for either the at-home or clinician-based treatment, the pre-wetting agent and/or BTX are applied to the skin surface (epidermis) prior to performing dermabrasion (such as microdermabrasion).

In some embodiments, the pre-wetting agent and/or BTX are applied without injection. Thus, the application is “needle-free” (non-invasive) in that the application of pre-wetting agent and/or BTX are not via subcutaneous or intradermal injection.

For example, the pre-wetting agent and/or BTX can be applied to the skin surface about 120, about 90, about 60, about 45, about 30, about 25, about 20, about 25, about 10, about 5 minutes or about 2 minutes prior to dermabrasion (such as microdermabrasion). This would be considered to be within one treatment period. The pre-wetting agent and/or BTX can alternatively be applied concurrently with dermabrasion (such as microdermabrasion). The pre-wetting agent and/or BTX can be applied at the time of dermabrasion, or immediately prior to dermabrasion (such as microdermabrasion).

In particular examples, a liquid form of the pre-wetting agent and/or BTX are applied substantially evenly across the surface of the skin that is to be subjected to dermabrasion, forming a layer of the pre-wetting agent and/or BTX on the skin. In one example, the pre-wetting agent and/or BTX are wet at the time of dermabrasion. Generally, the pre-wetting agent and/or BTX are not removed prior to performing dermabrasion. Thus, in one example, the skin in not washed between the application of pre-wetting agent and/or BTX, and the performance of dermabrasion (such as microdermabrasion).

Hyaluronic acid is a natural high viscosity mucopolysaccharide with alternating β (1-3) glucuronidic and β (1-4) glucosaminidic bonds. It has been prepared as a salt, such as a sodium salt. It is understood that the term hyaluronic acid includes its derivatives and broadly refers to naturally occurring, microbial and synthetic derivatives of acidic polysaccharides of various molecular weights constituted by residues of glucuronic acid and N-acetyl-D-glucosamine (see U.S. Pat. No. 6,573,249). Hyaluronic acid for use in the methods described herein is generally in a form suitable for topical administration to the skin, and can include salts, homologues, analogues, derivatives, complexes, esters, fragments, or sub-units of hyaluronic acid. Hyaluronic acid can be extracted from various natural tissues, such as cocks' combs, or also from bacteria such as Streptococcus species. In one example, hyaluronic acid is isolated from a Streptococcus, such as Streptococcus zooepidemicus. In another example, hyaluronic acid is extracted from human tissues. Hyaluronic acid can also be prepared by microbiological methods. In general, the molecular weight of hyaluronic acid is in the range of 50,000 to 8×10⁶ daltons, depending on the source and method of preparation.

In some embodiments, the hyaluronic acid (for example hyaluronic acid and the sodium salt thereof) has a molecular weight less than about 750,000 daltons (for example about 150,000 to about 225,000 daltons). Various grades of hyaluronic acid have been obtained. A preparation with an allegedly high degree of purity and alleged to be entirely free from side effects, is a non-inflammatory form described in U.S. Pat. No. 4,141,973; this preparation is said to have a molecular weight exceeding 750,000 daltons, preferably exceeding 1,200,000 daltons and is suggested for therapeutic use in various articular conditions. U.S. Pat. No. 4,636,524 discloses cross-linked gels of hyaluronic acid, alone and mixed with other hydrophilic polymers and containing various substances or covalently bonded low molecular weight substances and processes for preparing them. For example, gels are disclosed wherein a sample of sodium hyaluronate or hyaluronic acid from any source is dissolved in dilute alkaline solution. The molecular weight of hyaluronic acid can be from 50,000 up to 8×10⁶ and even higher. The alkali concentration in the reaction mixture can be from 0.005M to 0.5M and higher. The lower limit is dictated by the desire to have the pH of the medium not lower than 9 and the upper limit by the undesired hydrolysis of hyaluronic acid in an alkaline solution.

The hyaluronic acid can be dissolved in water, for example, in the form of a water-soluble sodium salt. Formulations containing hyaluronic acid for topical use are disclosed in U.S. Pat. No. 4,736,024; U.S. Pat. No. 4,636,524; U.S. Pat. No. 5,824,658; U.S. Pat. No. 6,136,793; U.S. Pat. No. 6,218,373. Examples of esters of hyaluronic acid include the methyl ester of a hyaluronic acid with a high molecular weight obtained by extraction from human umbilical cords as described in Jeanloz et al. (1950) J. Biol. Chem. 186, 495-511 and Jager et al. (1979) J. Bacteriology 1065-1067 and those described in U.S. Pat. No. 4,851,521. In one example, hyaluronic acid is diluted in a pharmaceutically acceptable carrier as about a 20% weight/volume to about a 30% weight/volume solution.

Chondroitin sulfate is also known as chonodroitinsufuric acid, and has a molecular weight of approximately 50,000 depending on the source and the method of isolation. This molecule is a high viscosity mucopolysaccharide (glycosaminoglycan) with N-acetylchondrosine as a repeating unit and with one sulfate per disaccharide unit. Chondroitin 4-sulfate and chondroitin 6-sulfate are two molecules of use in the methods disclosed herein. Other mucopolysaccharides, or modified mucopolysaccharides, may also be used in the methods described herein.

Collagen is a polypeptide substance that has a molecular weight of about 130,000. Collagen can be produced from bones and from body parts such as rat tail. Isolation of collagens has been described, for example, in U.S. Pat. No. 2,979,438. Collagen can also be prepared using genetic engineering methods. Generally, collagen is a molecule containing three polypeptide chains, alpha chains arranged in a triple helical conformation. The amino acid sequence of the alpha chain is predominately a repeating structure with glycine in every third position and proline or 4-hydroxyproline frequently preceding the glycine residues. In one embodiment, human collagen is utilized.

BTX is a toxin produced by the bacterium Clostridium botulinum and reversibly paralyzes striated muscle when administered in sub-lethal doses. It is understood that the term BTX includes BTX obtained from a natural source or made by synthetic means. BTX for use in the methods described herein is generally in a form suitable for needle-free administration to the skin. BTX is a generic term covering a family of toxins produced by C. botulinum comprising up to eight serologically distinct forms (A, B, C₁, C₂, D, E, F and G), each of which is distinguished by neutralization with type-specific antibodies. These toxins are among the most powerful neuroparalytic agents known (c.f. Melling et al., Eye 2:16-23, 1988). Serotypes A, B and F are the most potent. Without being bound by theory, the mode of action is believed to be an inhibition of the release of acetylcholine by the presynaptic nerve.

The different serotypes of BTX vary in the animal species that they affect and in the severity and duration of the paralysis they evoke. For example, it has been determined that BTX type A is 500 times more potent, as measured by the rate of paralysis produced in the rat, than is BTX type B. Additionally, BTX type B has been determined to be non-toxic in primates at a dose of 480 U/kg which is about 12 times the primate LD₅₀ for BTX type A (Moyer et al., “BTX Type B: Experimental and Clinical Experience,” in Therapy With Botulinum Toxin, Marcel Dekker, Inc Jankovic et al. (eds.), pgs. 71-85, 1994).

In one embodiment, BTX type A can be obtained by establishing and growing cultures of Clostridium botulinum in a fermenter and then harvesting and purifying the fermented mixture in accordance with known procedures. Alternatively, the BTX serotypes can be initially synthesized as inactive single chain proteins which must be cleaved or nicked by proteases to become neuroactive. High quality crystalline BTX type A can be produced from the Hall A strain of Clostridium botulinum. The Schantz process can be used to obtain crystalline BTX type A (see Schantz et al., Microbiol Rev. 56; 80-99, 1992). Generally, the BTX type A complex can be isolated and purified from an anaerobic fermentation by cultivating Clostridium botulinum type A in a suitable medium. This process can be used, upon separation out of the non-toxin proteins, to obtain pure BTXs, such as for example: purified BTX type A with an approximately 150 kD molecular weight, purified BTX type B with an approximately 156 kD molecular weight and purified BTX type F with an approximately 155 kD molecular weight.

BTXs and/or BTX complexes can be obtained from List Biological Laboratories, Inc., Campbell, Calif.; the Centre for Applied Microbiology and Research, Porton Down, U.K.; Wako, Osaka, Japan; Metabiologics, Madison, Wis.) as well as from Sigma Chemicals, St Louis, Mo. BTX-A serotype is available commercially for pharmaceutical use under the trademarks BOTOX™ (Allergan, Inc., Irvine, Calif., U.S.A.) and DYSPORT™ (Speywood Pharmaceuticals, Ltd., Maidenhead, U. K.). BOTOX™ consists of a purified BTX type A complex, albumin and sodium chloride packaged in sterile, vacuum-dried form. Specifically, each vial of BOTOX™ contains about 100 Units (U) of Clostridium botulinum toxin type A purified neurotoxin complex, 0.5 milligrams of human serum albumin and 0.9 milligrams of sodium chloride in a sterile, vacuum-dried form without a preservative. DYSPORT™ is sold in 500 Unit vials. BTX-A is roughly tenfold less toxic than BOTOX™ and approximately fourfold greater amounts of the DYSPORT™ product can be administered to achieve the same result as would be obtained using a specific number of Units of BOTOX™. For cosmetic uses, the vial contents can be diluted with a 1 or 2 ml of vehicle, such as sterile saline solution or the pre-wetting agent, which for BOTOX™ provides a 100 or 50 Unit/ml dilution. (DYSPORT™ can also be utilized.) For example, BOTOX™ can be reconstituted with sterile, non-preserved saline prior to administrated to a skin surface. To reconstitute vacuum-dried BOTOX™, a sterile solution (such as 0.9% Sodium Chloride or the desired wetting agent) can be used by drawing up the proper amount of diluent in the appropriate size syringe. Since BOTOX™ may be denatured by bubbling or similar violent agitation, the diluent is gently injected into the vial. For sterility reasons BOTOX™ is preferably administered within four hours after the vial is removed from the freezer and reconstituted. During these four hours, reconstituted BOTOX™ can be stored in a refrigerator at about 2° C. to about 8° C. Reconstituted, refrigerated BOTOX™ has been reported to retain its potency for at least about two weeks (see Neurology, 48:249-53:1997).

The pre-wetting composition including, for example, the hyaluronic acid, chondroitin sulfate or collagen can be formulated separately from the BTX composition. Alternatively, these two compositions can be formulated into one composition for simultaneous application of the pre-wetting agent and BTX composition. In some examples, the BTX composition is applied to the skin surface without the pre-wetting agent. In other examples, the BTX composition is applied to the skin with agents that facilitate transdermal delivery.

The pre-wetting and/or BTX compositions, or the combination thereof, can also contain additional substances that are customarily used in cosmetics, for example, perfume; antimicrobial agents; antibacterial agents; refatting agents; complexing and sequestering agents; pearlescent agents; plant extracts; vitamins, such as retinol or vitamin C; active agents; preservatives; bactericides; surfactants, dyes, colorants, pigments, or any substances which have a coloring effect; emulsifiers; thickeners; softening, moisturizing, and/or humectant substances; or other common constituents of a cosmetic or dermatological formulation, such as alcohols, polyols, polymers, foam stabilizers, electrolytes, organic solvents, or silicone derivatives. The compositions can also comprise functional additives such as keratolytic agents, oxidizers, sun-protection agents, and skin smoothing agents. The compositions may also contain components that are considered beneficial in mesotherapy injectional treatment of the skin and underlying subcutaneous tissue, including antioxidants, such as dimethylaminoethanol, alpha lipoic acid, and ascorbic acid. Agents that may enhance the efficacy of BTX may also be included in the compositions. Agents that may enhance the vascular perfusion of the skin, such as aminophylline, or pentoxifylline may also be included in the compositions. Agents that may enhance the turgor and tonicity of the skin as well as allow for the contraction or shrinkage of the underlying subcutaneous tissue structure, such as phosphatidyl choline and deoxycholate sulfate may also be included in the compositions. Physiologic substances that may provide hormonal benefit, such as substances of estrogenic or testosterrogenic stimulus to the skin, including estriol, and testosterone may also be included in the compositions.

The pre-wetting and/or BTX compositions, or the combination thereof, can include one or more preservatives. These preservatives include, for example, formaldehyde donors (such as, for example, DMDM hydantoin, which is available under the trade name GLYDAN® from Lonza), iodopropyl butylcarbamates (for example, those which are available under the trade names GLYCACIL-S™ from LONZA™ and/or DEKABEN LMB™ from Jan Dekker), parabens (for example, alkyl esters of the p-hydroxybenzoic acid, such as methyl-, ethyl-, propyl-, and/or butylparaben), phenoxyethanol, ethanol, benzoic acid, and salicylic acid. The preservation system can further comprise preservative auxiliaries, such as octoxyglycerin or glycine soya. Other preservatives or preservative auxiliaries include dibromocyanobutane (2-bromo-2-bromomethylglutarodinitrile), 3-iodo-2-propynyl butylcarbamate, 2-bromo-2-nitropropane-1,3-diol, imidazolidinyl urea, 5-chloro-2-methyl-4-isothiazolin-3-one, 2-chloroacetamide, benzalkonium chloride, and benzyl alcohol.

The compositions can also include one or more conditioners. Such conditioners can include, for example, any compound listed in the International Cosmetic Ingredient Dictionary and Handbook (Volume 4, Publishers: R. C. Pepe, J. A. Wenninger, G. N. McEwen, The Cosmetic, Toiletry, and Fragrance Association, 9.sup.th edition, 2002) in Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive, and Skin Protectants, as well as any compound listed in EP 0934956 (pp. 11-13) under water soluble conditioning agent and oil soluble conditioning agent. Other conditioners include, for example, compounds that are called polyquaternium in accordance with the International Nomenclature for Cosmetic Ingredients (INCI), in particular Polyquaternium-1 to Polyquaternium-56.

The compositions can be dispensed from a soft tube, a jar, a bottle, a pump, a can, a spray can or spray bottle, or from some other known container.

Dermabrasion performed by a clinician can be practiced by any method known in the art, including particle-free or particle-based dermabrasion, and including any method of microdermabrasion. Thus, in an additional embodiment, particle-free dermabrasion is utilized.

Particle-free methods are known in the art and include, but are not limited to, VIBRADERM™ and DIAMOND TOME™ (see U.S. Pat. No. 6,241,739). In this example, the device for microdermabrasion comprises a hollow tube with an abrasive material permanently attached to a skin contacting end. The abrasive coated tip is moved over the skin surface while a vacuum is applied through the tube to the skin surface to remove cells abraded from the skin surface. The vacuum also causes the skin to be held in intimate contact with the abrasive tip during the treatment procedure.

Other microdermabrasion methods include, but are not limited to, POWER PEEL® (see U.S. Pat. No. 5,037,432). In this method, the apparatus is usable to remove surface portions of human tissue in an adjustable manner and essentially comprises a tool provided with a supply tube along which abrasive reducing substances are conveyed under pressure. A throughhole in the head disposed along the axis of the tube permits the substances to abrade the region of tissue facing the hole. A collection tube in which is created a depression is provided for the purpose of removing under suction both the reducing substances and the portions of tissue removed during the treatment.

Another exemplary, non-limiting method of a dermabrasion system for use in the methods disclosed herein is DERMA GENESIS™ Particle Skin Resurfacing, SMARTPEEL®, DERMAGLIDE™ Systems, MEGA PEEL™ Systems, or ULTRAPEEL™ by Mattioli Engineering. In one example, these methods include the use of a mixing bottle of air and reducing substances in dermabrasion treatments, wherein the mixing bottle is a disposable sterilized substantially cylindrical body. The mixing bottle includes the reducing substances and is provided with connections respectively to connect to a valve and a pneumatic duct leading to a handle, one of the connections extending inside the mixing bottle with a suction tube provided with a hole through which the reducing substances enter into the pneumatic duct. In these devices the mixing bottle is filled with the reducing substances in a sterilized environment and then sealed by connection plugs (see U.S. Pat. Nos. 5,810,842 and 5,954,730).

Dermabrasion can also include the use of abrasive tipped devices or rotating brushes and cylinders coated with abrasive particles, such as diamond dust, have been used to remove skin layers (U.S. Pat. No. 2,712,823; U.S. Pat. No. 2,867,214; U.S. Pat. No. 2,881,763; and U.S. Pat. No. 2,921,585). U.S. Pat. No. 5,800,446 describes a stick, glove finger tip or glove palm coated with an abrasive material which is rubbed over the skin surface to provide a polishing action. U.S. Pat. No. 3,964,212 directed to a pneumatic grinding machine for flat surfaces, incorporates a rotating grinding tool enclosed in a housing with air flowing over the surface to collect dust created by the grinding operation.

U.S. Pat. No. 4,378,804 is directed to a skin abrasion device that uses flowing water to rotate an abrasive brush and create a vacuum to remove loosened skin particles. The rotating brush is usually used in conjunction with a liquid detergent or medicinal compound applied to the skin surface being scrubbed. Chemicals, ultrasonic oscillating tips (U.S. Pat. No. 5,012,797) and lasers have also been used for a more aggressive abrasion. U.S. Pat. No. 5,037,431 describes the use of a pressurized jet of a liquid, such as water or sterile saline, to fragment and remove diseased tissue without harming surrounding healthy tissue. This device operates in conjunction with vacuum aspiration to remove the liquid and fragmented tissue.

Chemical methods of dermabrasion involve the use of pharmaceutical agents, such as alpha hydroxy acids (AHA), such as glycolic, lactic or fruit acid. Various concentrations of an AHA can be applied weekly or at longer intervals to obtain the best result. A trichloroacetic acid (TCA) application can also be utilized.

In one embodiment, the method further includes iontophoresis treatment. Methods for performing iontophoresis on a patient are well known in the art. See, for example, U.S. Patent Application Publication No. 2005/0107832, U.S. Patent Application Publication No. 2005/0049642, U.S. Patent Application Publication No. 2004/0220622, U.S. Patent Application Publication No. 2004/0015190, U.S. Patent Application Publication No. 2003/0187478, U.S. Patent Application Publication No. 2003/0014081, and U.S. Patent Application Publication No. 2002/0147467. These applications disclose the use of iontophoresis for enhanced drug delivery to the skin. It is disclosed herein that the use of at least 10 units of BTX, such as about 20 units to about 200 units of BTX, about 30 to about 100 units, or about 30 units to about 50 units, including 15 units, 20 units, 25 units, 30 units, 40 units, 50 units, 60 units, 70 units, 80 units, 100 units, 120 units, 150 units, 200 units, can be used either alone or in combination with at least about 20% of the pre-wetting agent, such as about 20% to about 30% of the pre-wetting agent, in conjunction with dermabrasion. In several examples, about 20 to 200 units of BTX can be used either alone or in combination with about 25%, about 26%, about 27%, about 28%, about 29% or about 30% pre-wetting agent (W/V), such as 25%, 26%, 27%. 28%, 29% or 30% pre-wetting agent (W/V) in conjunction with dermabrasion for an unexpectedly superior effect on skin texture, such as a reduction in lines and wrinkles. The application of the pre-wetting agent and/or BTX can be used in conjunction with, or in the absence of iontophoresis.

The methods can also utilize particle-based dermabrasion, including particle-based microdermabrasion. Specific, non-limiting examples of these procedures are PARISIAN PEEL® Medical Microdermabrasion Treatment, and DERMAGLOW™ Particle Skin Exfoliation, which utilize aluminum oxide. Generally, the application of pre-wetting agent can be used with any method of dermabrasion (and any method of microdermabrasion). However, the application of the pre-wetting agent in combination with BTX is generally used only with particle-free dermabrasion, such as particle-free microdermabrasion.

Generally, particle-based dermabrasion includes the use of particles as abrasive agents. For example, the skin surface can be abraded using powdered aluminum oxide particles or a liquid topical composition containing suspended aluminum oxide (see U.S. Pat. No. 4,957,747). In another example, U.S. Pat. No. 5,037,432 discloses the use of pressurized delivery, using compressed air, of a powdered, abrasive substance and the removal of the abrasive substance and loosened skin tissue using a vacuum. The abrasive substance is typically microcrystals of quartz, metal, or aluminum oxide. The powdered abrasive is blown through a wand which has a hole in the skin contact end to provide access of the abrasive to the skin surface being treated. An alternative is to cause the aluminum oxide powders to flow by applying a vacuum to the exhaust side of a container holding the abrasive powder and entraining the powder in a flowing gas stream. The powder is then drawn by the vacuum through a treatment tool, across the skin surface to abrade or rub off the epidermis and then recovered along with the skin particles in a collection chamber (U.S. Pat. No. 5,100,412; U.S. Pat. No. 5,207,234; U.S. Pat. No. 5,810,842).

The process of mechanical disruption of the skin surface as described herein may also be performed by using mechanical abrasive applications to the skin, such as micronized grit or scrub particle applications which exfoliate and desquamate the outer skin surface. Mechanical disruption of the skin surface can also be achieved by light fraise or gritted paper abrasion. Methods for mechanical disruption of the skin surface include, but are not limited to, particle-assisted surface disruption, chemoexfoliation, iontophoretic disruption, vibrational disruption, such as ultrasound, photo disruption of the skin, and vacuum-assisted disruption.

The abrasive composition includes an abrasive agent that is capable of abrading a skin surface. In several embodiments, this abrasive substance can be, for example, a crystal, such as magnesium oxide crystals, aluminum oxide crystals, sodium chloride crystals, other salt crystals, or baking soda crystals, or any biocompatible or inert particulate material. The abrasive substance can also be, for example, a powder, such as sodium bicarbonate powder. The abrasive agent can be a combination of any of the above substances. Sodium bicarbonate is an effective abrasive or skin-abrading material that does not impart a harsh, scratchy feel. A combination of sodium chloride crystals and sodium bicarbonate powder provides an effective skin-abrading system for dermabrasion while minimizing abrasive irritation and harshness likely to cause skin damage from over- or improper use. Thus, the combination of sodium chloride crystals and sodium bicarbonate powder is well suited for us with dermabrasion treatment. The abrasive composition (comprising the abrasive agent) can be in the form of a lotion, gel, cream, or in an ointment or oil base, or in a sprayable liquid form. The abrasive agent can be an agent suitable for in-home use, or use by a clinician.

The abrasive composition including the abrasive agent, can include, for example, about 2-50% abrasive particles by weight, such as about 2-30% abrasive particles by weight, about 5-25% abrasive particles by weight, or about 10-20% abrasive particles by weight. The abrasive particles can have, for example, a mean particle size distribution of about 20 to about 500 microns, such as about 50 to about 400 microns, such as about 75 to about 350 microns, such as about 100 to about 300 microns, or about 50 to about 2000 microns, such as about 75 to about 1500 microns, such as about 100 to about 1000 microns, such as about 125 to about 900 microns, such as about 150 to about 850 microns.

Wetted dermabrasion kits are also disclosed herein for use in the home or by a clinician. In one example, the wetted dermabrasion kit includes the pre-wetting composition and/or the BTX agent, either in two separate containers or as a single composition in a single container. In one example, this kit is suitable for use by a clinician. In such example, the wetted dermabrasion kit can further include applicators or tools, such as applicators or tools to assist with the particle-free dermabrasion treatment. Both the applicator and the tool can be included in the kit. Generally the kit also includes instructions for use. These instructions can written or in a digital formal (such as a videotape, DVD or CD) for use with electronic devices such as computers, CD players, mp3 players or DVD players.

In some example, the wetted dermabrasion kit includes the pre-wetting composition and the abrasive composition, either in two separate containers or as a single composition in a single container. In one example, this kit is suitable for use in the home. The wetted dermabrasion kit can further comprise an applicator, such as a sponge or cloth, for applying the pre-wetting or abrasive compositions. Alternatively, one or more fingers can be used to apply the compositions. In another example, the kit can include a tool to assist with the dermabrasion treatment, such as an applicator that functions to massage the abrasive composition into the surface of the skin. Both the applicator and the tool can be included in the kit. Generally the kit also includes instructions for use. These instructions can be written or in a digital formal (such as a videotape, DVD or CD) for use with electronic devices such as computers, CD players, mp3 players or DVD players.

In one example, the pre-wetting composition and abrasive composition are applied to a target skin area for dermabrasion treatment. Following a treatment, the compositions can be rinsed away from the skin with ordinary tap water. The pre-wetting composition, the abrasive composition, or the combined composition can contain components to aid or promote dispersion of the agent in water to facilitate rinsing of the skin free of the compositions. Also, the abrasive components in the abrasive composition can be water soluble, and therefore can be dissolved and rinsed away so as to prevent leaving any abrasive particulate or particulate residue on the treated skin following dermabrasion.

In one specific non-limiting example, the wetted dermabrasion kit can be used by a person in the home as follows. First, a measured quantity of the pre-wetting composition is applied to the surface the skin, followed by application of the abrasive composition, by the use of an applicator, such as a sponge or cloth, or by the use of one or more fingers, to provide an even layer of the composition on the skin surface, avoiding the eye area. The pre-wetting composition and abrasive composition can alternatively be provided together in one composition that is applied to the skin surface. The skin surface is then massaged, taking care to avoid contact with one's eyes, for example, by gently rubbing the composition with the fingertips, applying light to medium pressure, in a circular motion, ensuring that the skin surface is continued to be “wetted” with the “wetted formulation. This can be done for a certain number of revolutions, such as about 1-8, about 2-6, about 3-4, or about 10-15. Alternatively, it can be done for a certain length of time, for example, for about 30 seconds, for about 1 minute, for about 1.5 minutes, or for about two minutes. Once wetted dermabrasion is complete, the composition can be washed away from the treated skin with ordinary tap water. Moisturizers can then be applied for the next 24 to 48 hours. Generally, normal skin care procedures, such as makeup application and cleansing, can be resumed immediately.

In some examples, a kit is provided that allows a user to apply topical BTX and/or the pre-wetting agent in between particle-free dermabrasion treatments at the clinic to prolong the effects generated by the particle-free dermabrasion.

Wetted dermabrasion (such as microdermabrasion) can be performed as described above bi-weekly or monthly, or for some other interval, such as once every 3 to 5 days. In one example, the wetted dermabrasion is not performed more often than bi-weekly.

Improving skin quality includes reversing, slowing the progression of, or preventing skin changes associated with natural or innate aging. As used herein, “prevent” and variations thereof refer to any degree of delaying the onset of skin changes. For example, improving skin quality includes the reversal, slowing the progression of, or prevention of skin changes associated with sun damage or photo aging—skin changes associated with exposure to sunlight or other forms of actinic radiation (for example, UV radiation and tanning booths). As another example, improving skin quality also can include reversing, slowing the progression of, or preventing skin changes resulting from extrinsic factors, including, but not limited to, radiation, air pollution, wind, cold, dampness, heat, chemicals, smoke, cigarette smoking, and combinations thereof. Improving skin quality also can include reversing, preventing or reducing scarring the can result, for example, from certain skin conditions (for example, acne), infections (for example, leishmaniasis), or injury (for example, abrasions, punctures, lacerations, or surgical wounds). Improvements to the skin can also include at least one of the following: making facial lines appear less noticeable, making facial lines and/or wrinkles feel plumped, improving the appearance of suborbital lines and/or periorbital lines, improving the appearance of crow's feet, reducing and/or diminishing the appearance of wrinkles, particularly facial wrinkles on the cheeks, forehead (for example, perpendicular wrinkles between eyes, horizontal wrinkles above the eyes), and/or around the mouth, and particularly deep wrinkles, folds, or creases, improving skin suppleness, reducing and/or eliminating fine and/or deep lines, folds and creases, and smoothing skin. Methods for measuring improved skin quality are known in the art. See, for example, U.S. Pat. Nos. 6,866,856 and 6,682,763.

Skin changes treatable by practicing the methods and using the kits disclosed herein include, for example, wrinkles (including, but not limited to, human facial wrinkles), creases, furrows, folds and fine lines, deepening of skin lines, thinning of skin, reduced scarring, yellowing of the skin, mottling, hyperpigmentation, appearance of pigmented and/or non-pigmented age spots, leatheriness, loss of elasticity, loss of recoilability, loss of collagen fibers, abnormal changes in the elastic fibers, deterioration of small blood vessels of the dermis, formation of solar increased visible vasculature on the skin surface, and combinations thereof.

Improving skin quality includes decreasing, reducing, and/or minimizing one or more of the skin changes discussed above. Improving skin quality can result in the skin having a more youthful appearance. Improving skin quality can result in the skin having a smoother, hydrated (less dry), or less scaly appearance. For example, in certain embodiments, improving skin quality can include a reduction in roughness, dryness, or scaliness. Improving skin quality includes the effacement and improvement of lines and wrinkles, improvement in turgor, and tonicity, with the observed desired effects of lifting and tightening.

The textural qualities of the skin can be improved, including softness, suppleness, and smoothness, leading to enhancement of luster, clarity and brightness. Additional and important qualities of the skin that can be subjectively and objectively measured include, but are not limited to skin laxity, or conversely skin tightness, and the presence and degree of textural fine lines and coarser lines within the skin.

These are the same qualities by which the external aspects of appearance (for example, aging of skin) are judged. Improvement in these qualities by the method of treatment and kits disclosed herein result in a benefit based on visual judgment of appearance. Changing a quality of the skin by the methods disclosed herein lessens the appearance of aging of the skin.

Desired benefits may include not only physiologic benefit to the skin, but therapeutic and pharmacologic benefits, such as possible malignancy prevention and treatment, whether by chemoprevention or enhancement of photodynamic therapy. Benefits may also include acne treatment and suppression, by including compositions which suppress sebaceous glandular activity, enhance bacterial suppression, or enhance retinoid delivery into the skin.

The percentage of improvement can be, for example, at least about 50%, at least about 60%, at least about 70%, at least about 80%, at least about 90% or at least about 100% compared to the baseline score prior to treatment with pre-wetting agent and the abrasive agent. Without being bound by theory, the gains can be due to an enhancement of the process of dermabrasion by enabling a saturation of the skin with the pre-wetting agent, for example, hyaluronic acid and/or BTX. In one embodiment, the improvement is at least about 80% compared to subjective score prior to treatment. In another embodiment, the improvement is at least about 100% compared to subjective score prior to treatment. The improvement can be measured by both subjective and objective methods, and can be quantified using a subjective scoring or a panel scoring, amongst other methods. By “baseline” is intended the score prior to treatment or the score on an untreated area of skin. In one example, a “subjective score” is intended a rating given to the treated and control areas by the patients themselves, subsequent to the treatment. In another example, a “panel score” is a score given by non-biased observers. This score can be determined, for example, by looking at photographs.

The entire method can be performed multiple times for optimal results. Thus, several applications of pre-wetting agent in conjunction with dermabrasion can be performed on a single subject. In one embodiment, the method is performed twice within a two week time period. In another embodiment, the method is performed weekly. In another embodiment, the method is performed monthly. In another embodiment, the method is performed at least once every one to two weeks. In a further embodiment, the method is performed at intervals of about every six to about eight weeks. For example, two, three, four, five, six, seven, eight, nine or ten treatments can be administered, wherein the individual treatments are separated by six to eight weeks. In yet another embodiment, the method is performed two to five times in a period of two to five weeks.

It must be noted that as used herein and in the appended claims, the singular forms “a”, “an”, and “the” include plural referents unless the context clearly dictates otherwise. Unless defined otherwise, all technical and scientific terms used herein have the same meaning as commonly understood to one of ordinary skill in the art to which this disclosure belongs. “Comprising” means “including.” All publications, patent applications, patents, and other references mentioned herein are incorporated by reference in their entirety.

Without further elaboration, it is believed that one skilled in the art can, using this description, utilize the present disclosure to its fullest extent. The following examples are illustrative only, and not limiting of the remainder of the disclosure in any way whatsoever.

### EXAMPLES

### Example 1

### Saline-Wetted Microdermabrasion with Iontophoresis Versus 30% Hyaluronic Acid-Wetted Microdermabrasion with Iontophoresis

Twelve patients were selected for this study group. The left periorbit and left upper lip were selected for treatment with 30% hyaluronic acid (30% HA) “wetted” particle free DIAMOND TOME™ microdermabrasion (PFMD) followed by TRANSDERM IONTO™ electroporation iontophoresis. Hyaluronic acid used in these studies was obtained from Apothe'Cure; the hyaluronic acid was purchased originally from Xenos Bioresources, Inc. located in Santa Barbara. As disclosed by Xenos Biosciences, Y-921 from Streptococcus zooepidemicus is cultured in an appropriate medium at 27° C. for 42 hours. The culture fluid was then treated with trichloroacetic acid, and centrifuged, and the supernatant was collected. Following alcohol precipitation at pH=7.0, the crude product was then refined further and diluted to 30% weight per volume. In the examples below, reference to “30% hyaluronic acid” refers to 30% hyaluronic acid weight per volume.

The right periorbit and right upper lip were selected for treatment as a control using saline “wetted” DIAMOND TOME™ microdermabrasion followed by TRANSDERM IONTO™ electroporation iontophoresis. The left cheek was treated as a control with the identical application of 30% hyaluronic acid (W/V) “wetted” particle free microdermabrasion as the left periorbit and left upper lip, but without TRANSDERM IONTO™ electroporation iontophoresis. The right cheek was treated as a control with the application of saline rather than hyaluronic acid “wetted” particle free micro-dermabrasion, but without TRANSDERM IONTO™ electroporation iontophoresis. Two treatment sessions were performed two weeks apart. The microdermabrasion time was two minutes. The volume of application was limited to that amount required to wet the surface of the skin.

The skin surface was “prewetted” with either 30% hyaluronic acid (W/V, Apothe'Cure Pharmacy compounding, streptococcal derived) or saline, and particle-free microdermabrasion with DIAMOND TOME™ was performed. In some cases, surface wetted 30% HA TRANSDERM IONTO™ was performed subsequent to the microdermabrasion.

Objective scoring was performed using the CANFIELD™ clinical photography platform. Since the lines and textural quality of the periorbit and cheeks were “washed out” with the standard lighting of full face flash, a method of cross axis lighting was developed to enhance the visualization of the desired right or left face. Objective scoring was performed by non-biased observers who rated the pictures on a graded scale (0-12) based on the presence of lines and wrinkles. This rating was performed blinded, or without knowledge as to whether the picture was of treated or control skin. Subjective scoring was performed by the patients themselves, on a graded scale (0-12). Average scores for treated or untreated (control) skin as well as percent change were determined.

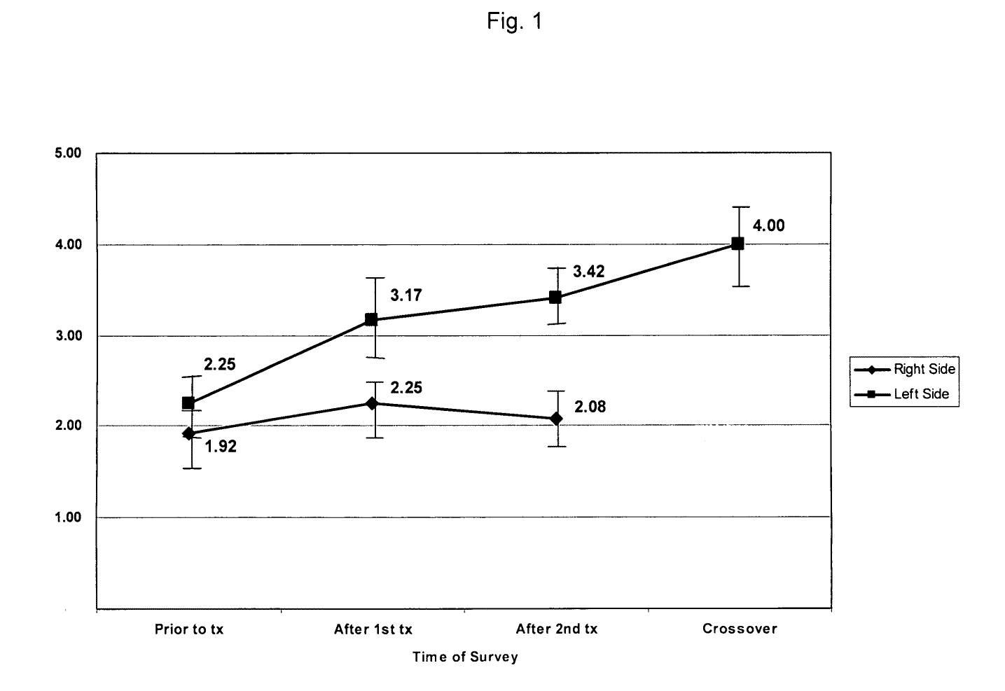

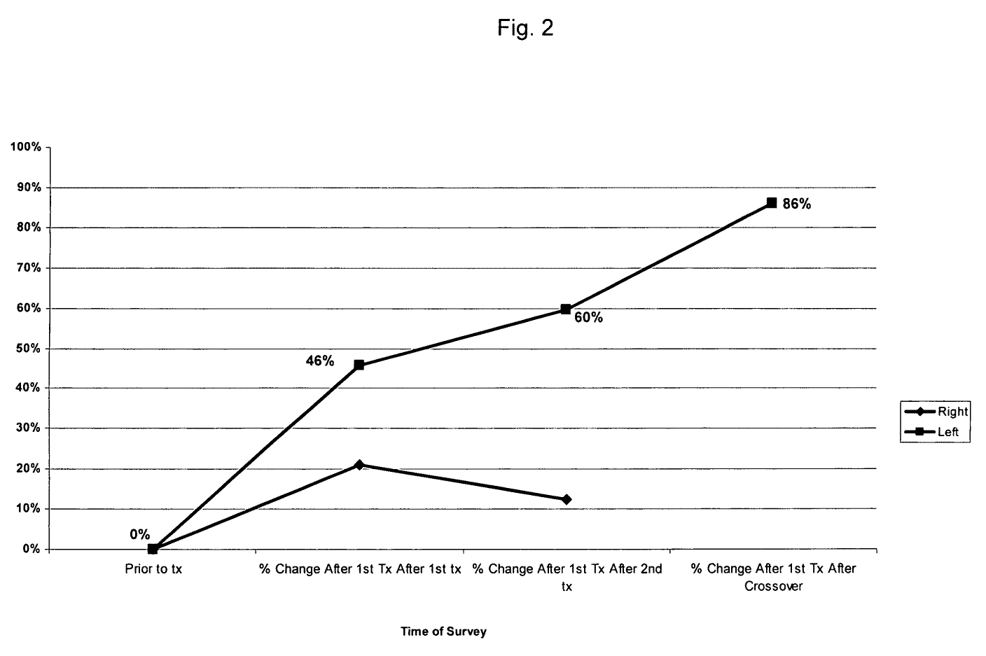

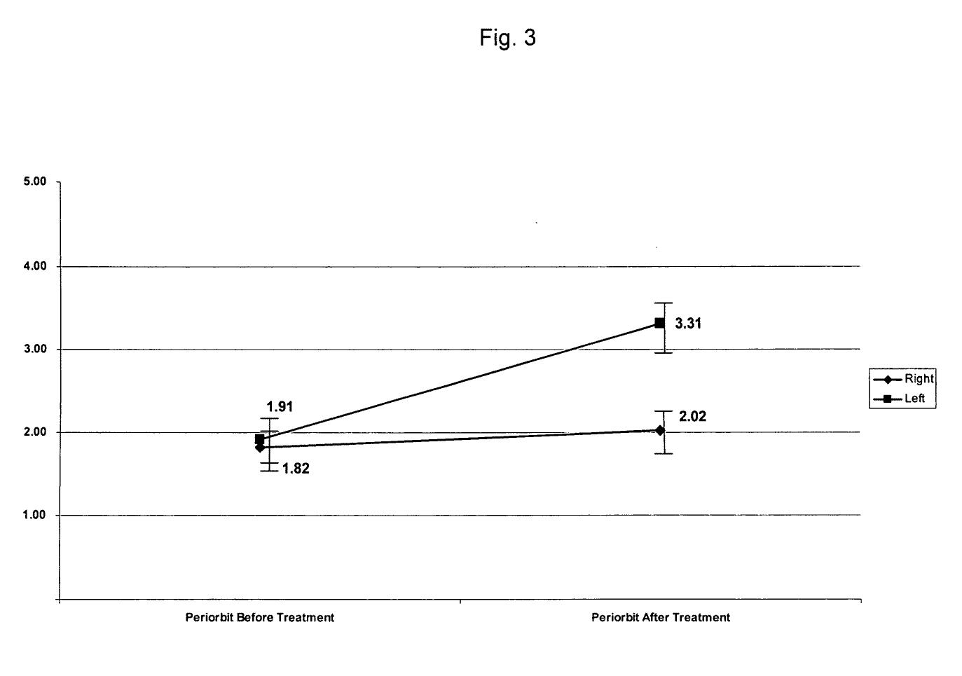

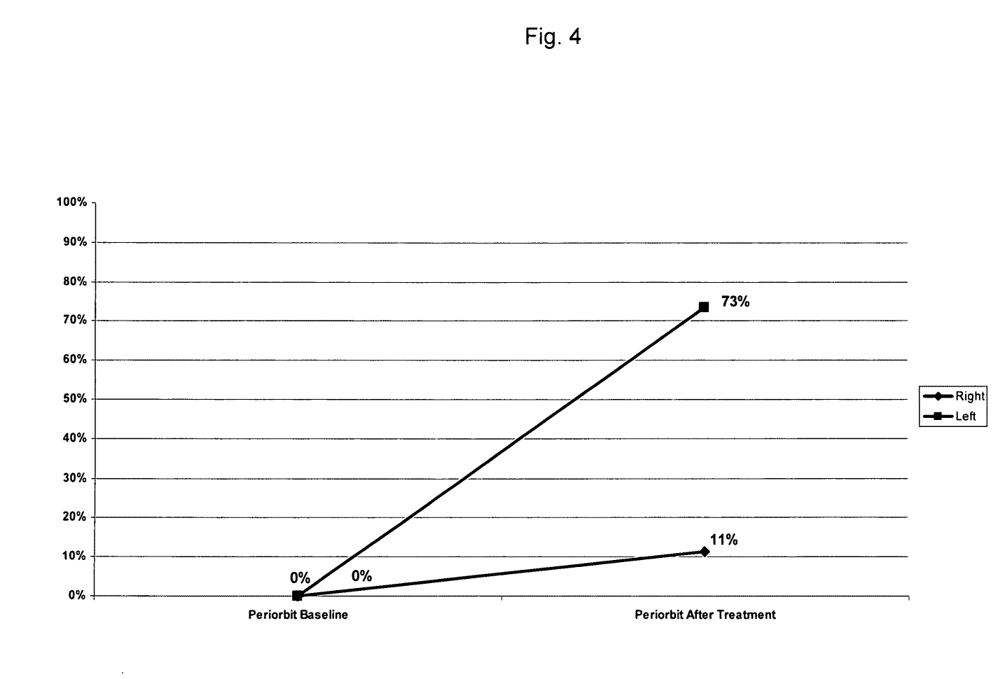

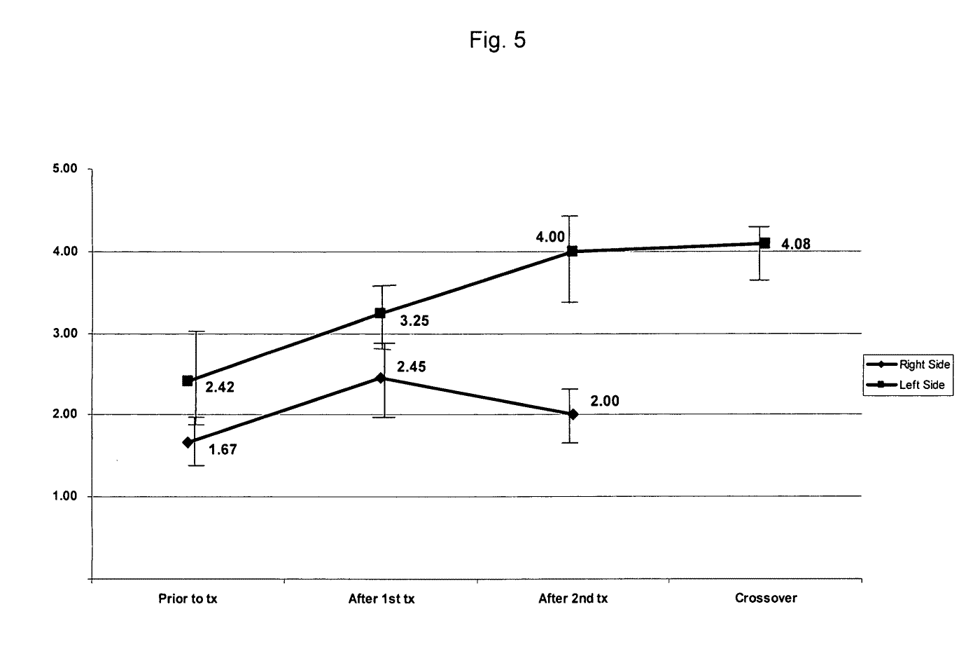

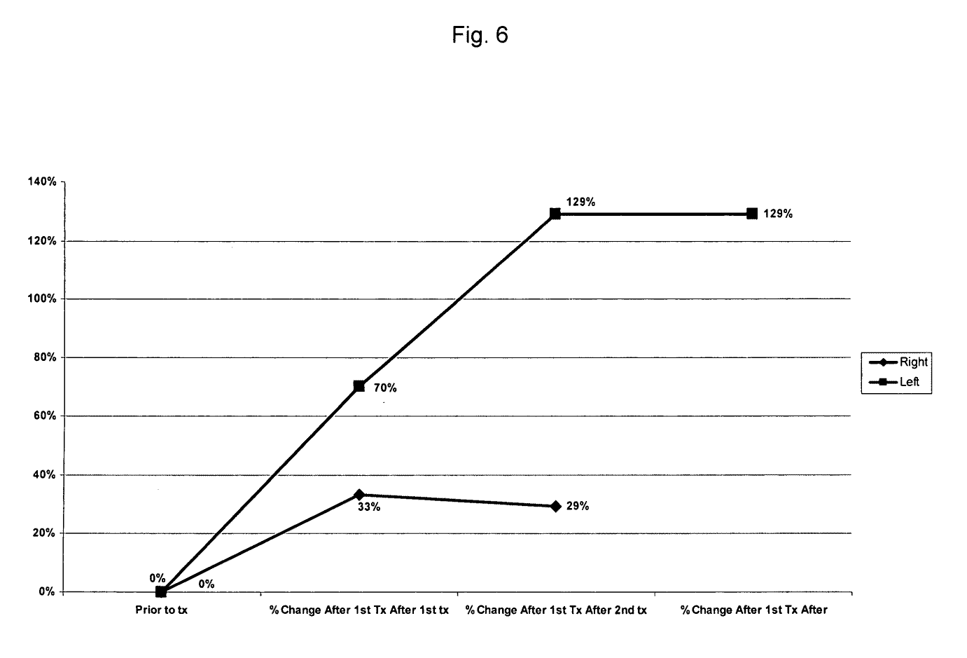

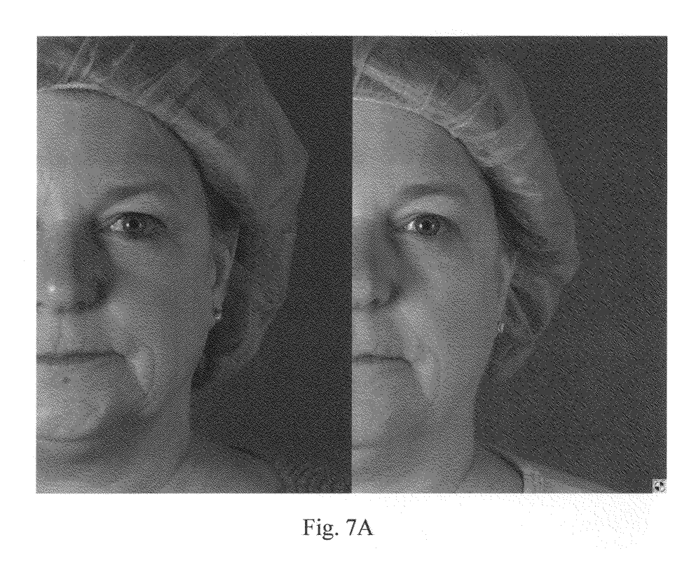

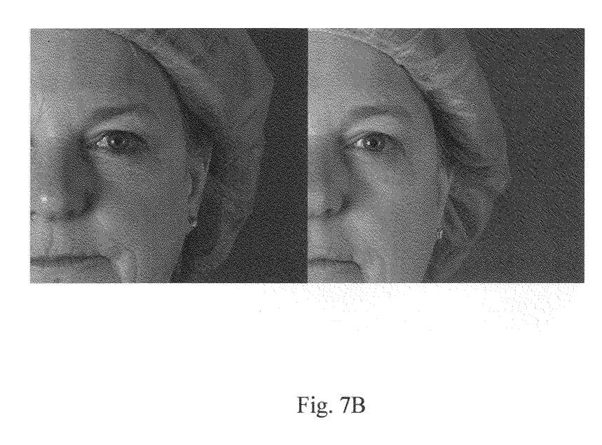

The subjective scoring of hyaluronic acid-wetted microdermabrasion followed by iontophoresis was higher than that of saline-wetted microdermabrasion followed by iontophoresis for the periorbit (FIG. 1, FIG. 2, and FIGS. 7A and B). Similarly, the objective scoring for hyaluronic acid treatment was higher than that for saline treatment followed by iontophoresis (FIG. 3, FIG. 4, and FIGS. 7A and B). The subjective scoring for the upper lip was also increased for hyaluronic acid-wetted microdermabrasion followed by iontophoresis (FIG. 5, FIG. 6, and FIGS. 7A and B).

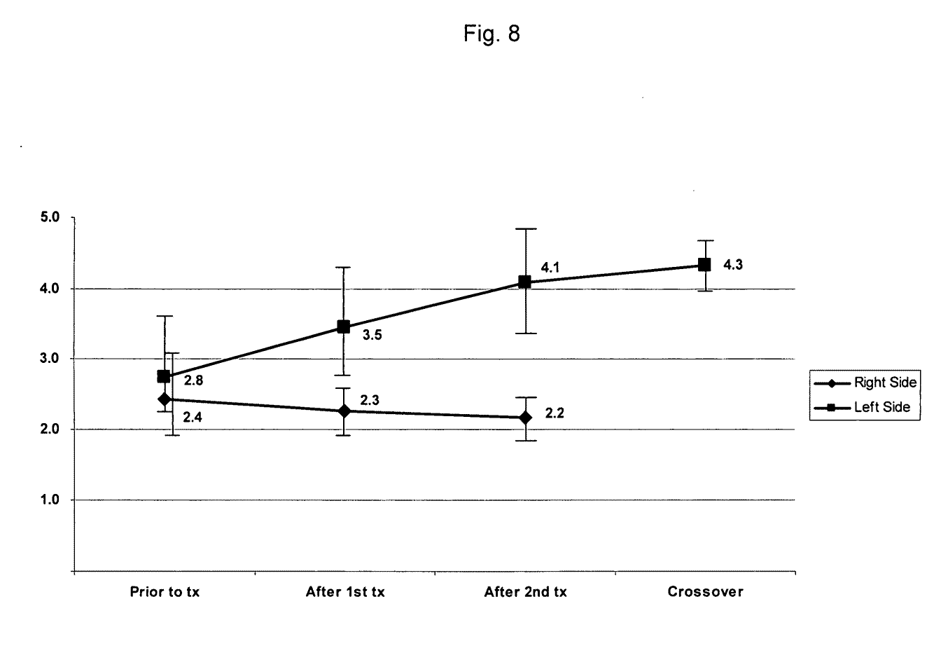

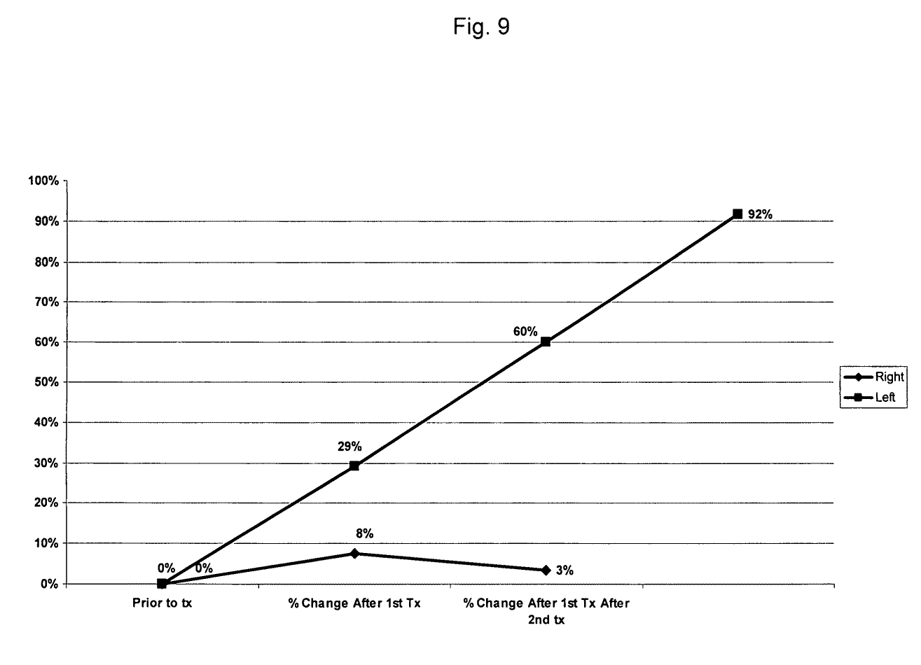

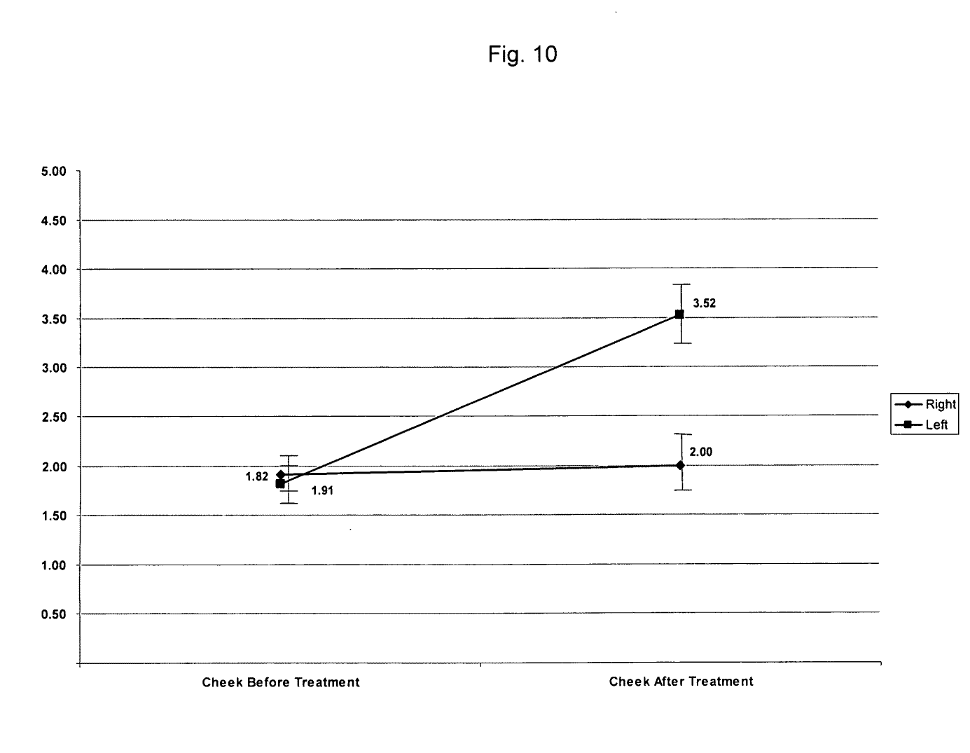

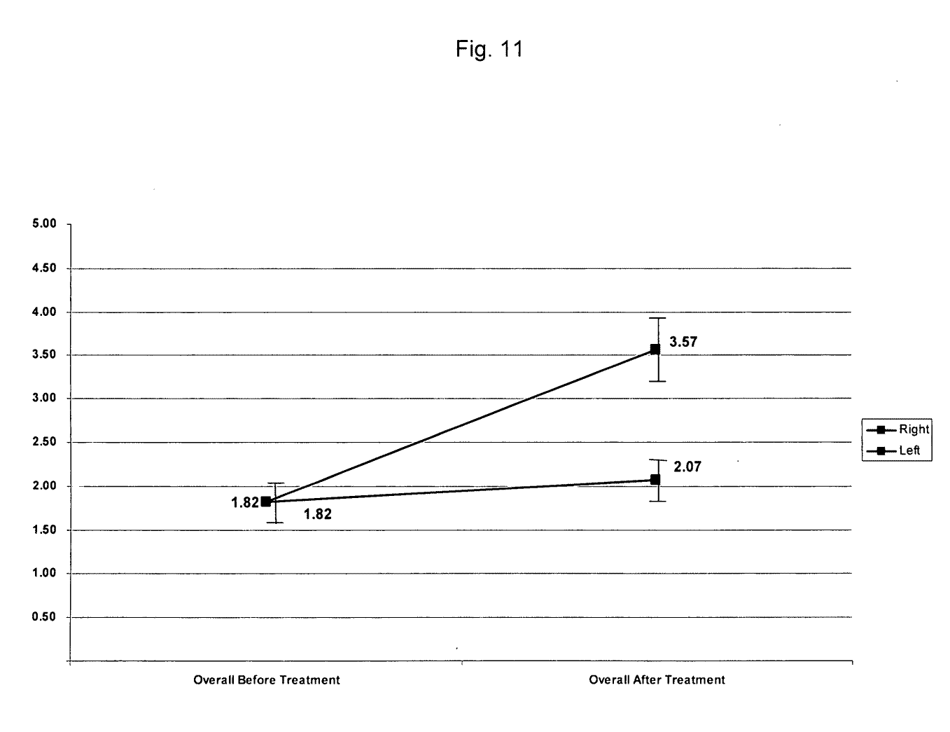

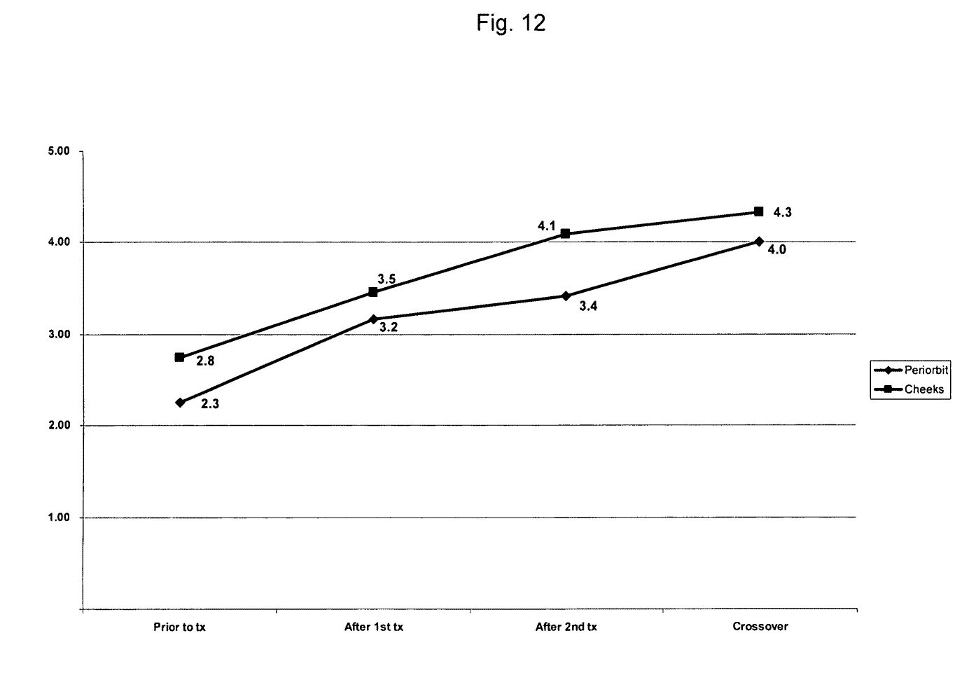

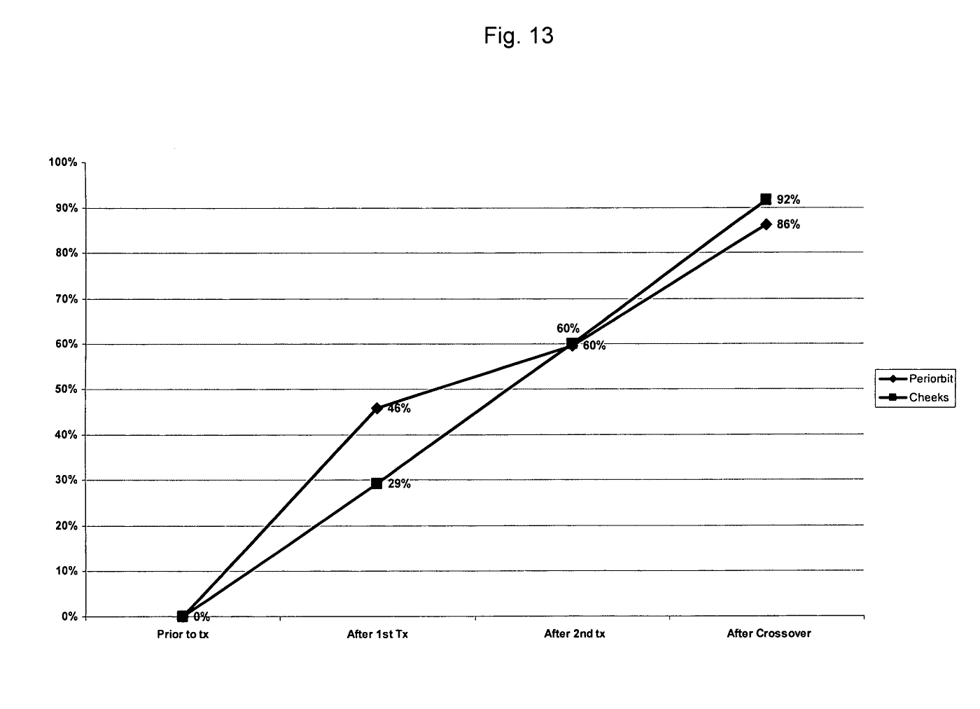

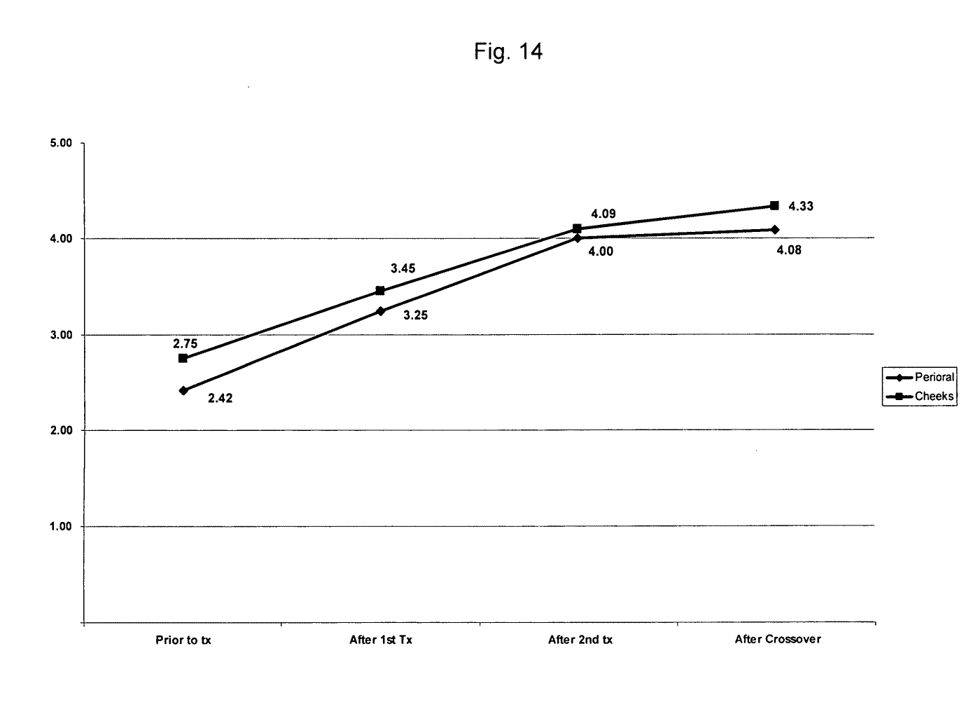

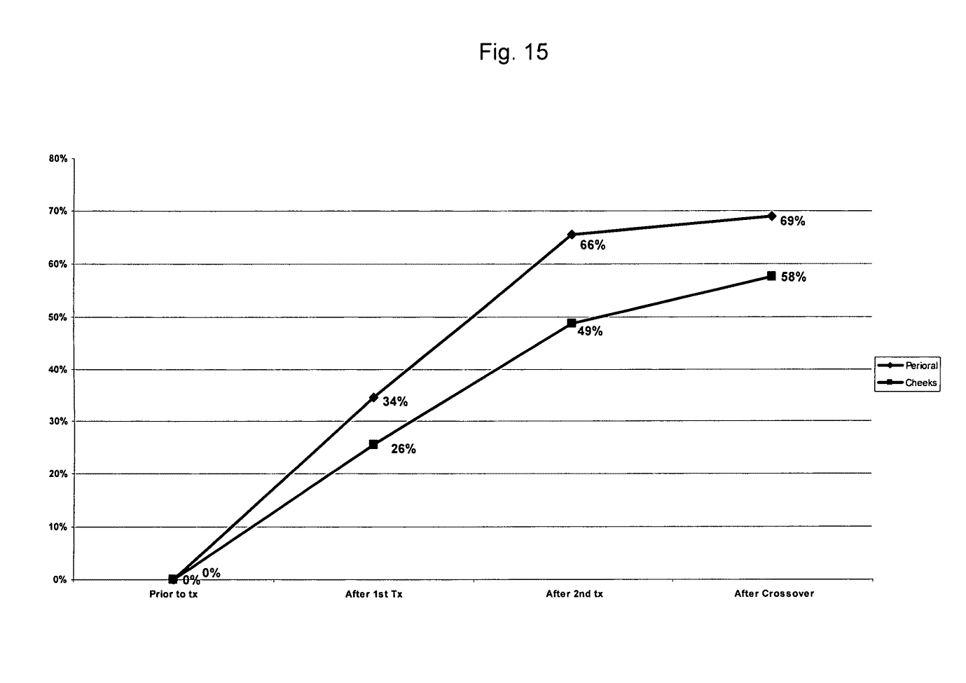

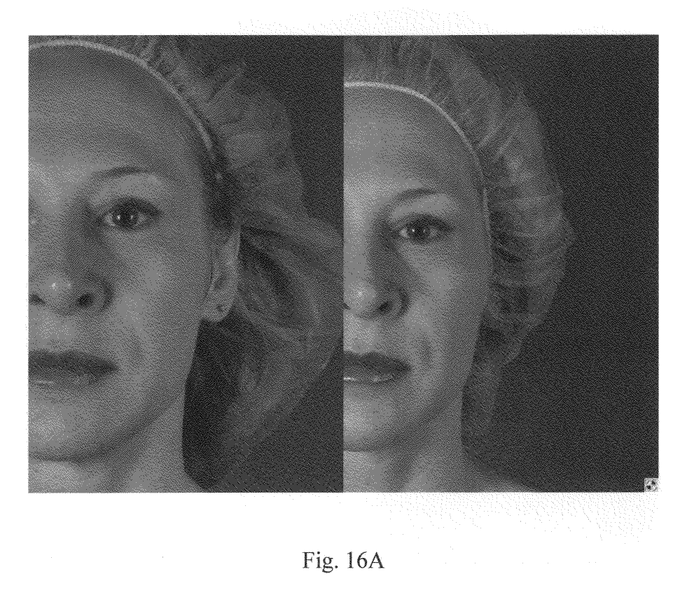

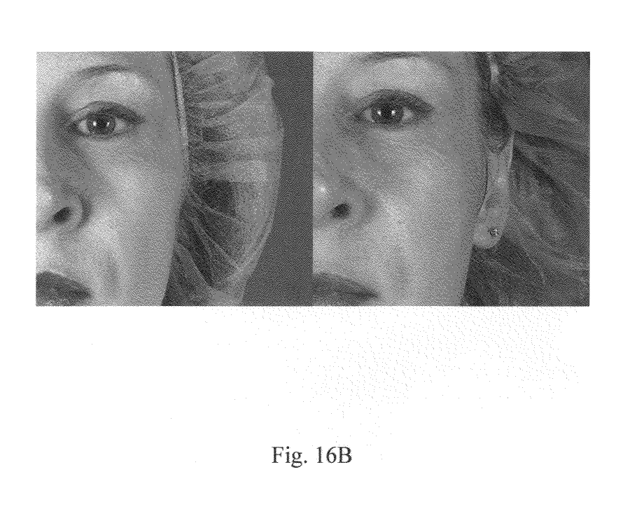

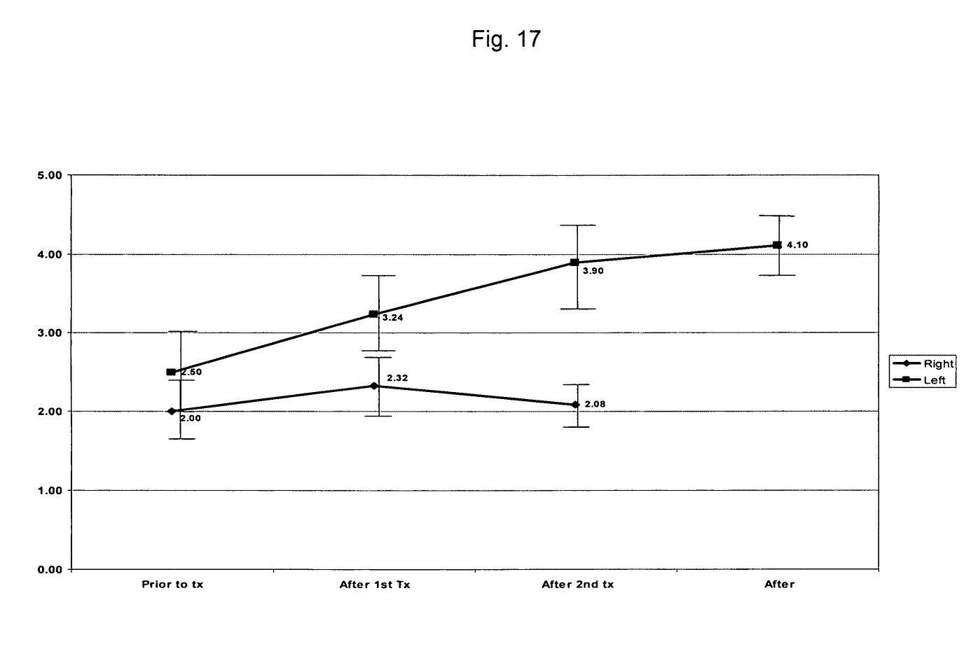

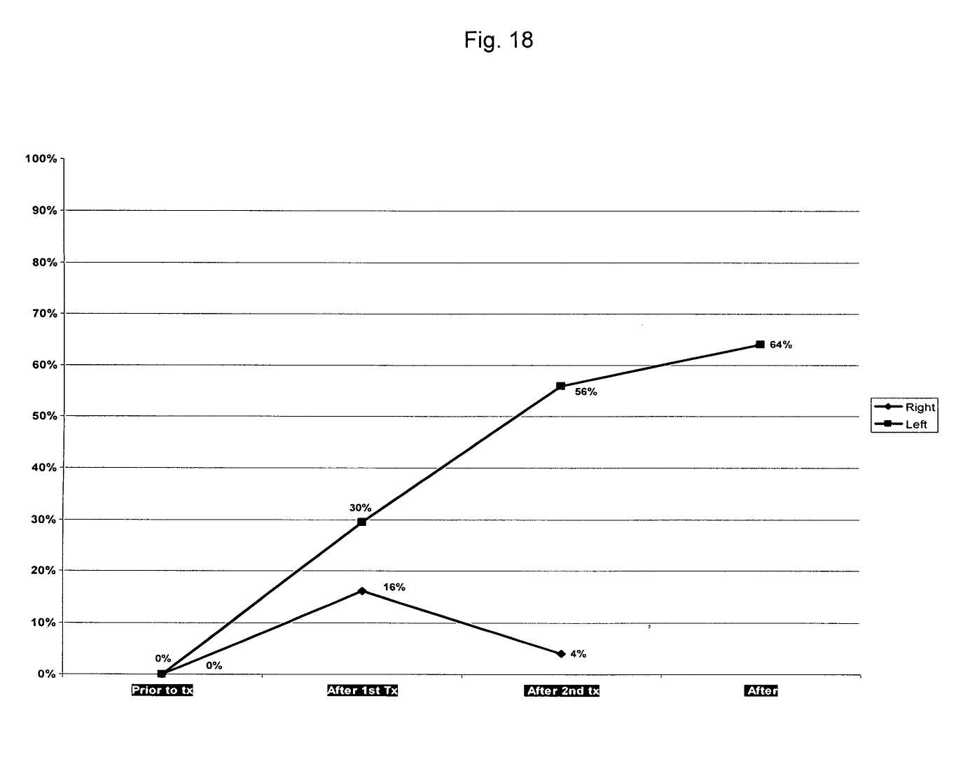

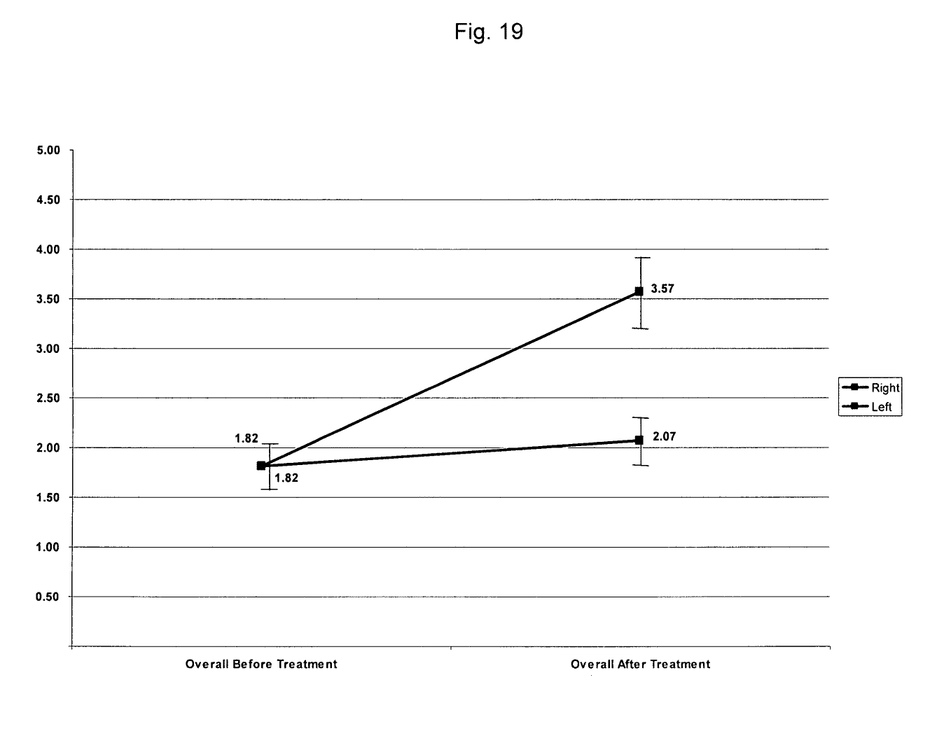

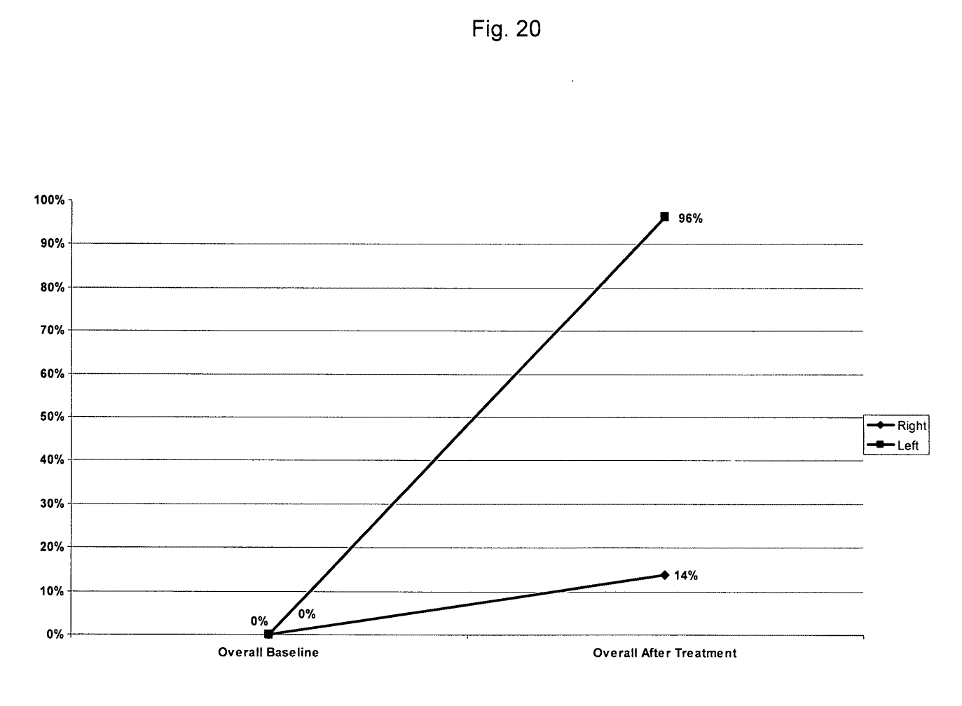

The cheek subjective and objective scoring was higher for hyaluronic acid-wetted microdermabrasion without iontophoresis than for control saline-wetted microdermabrasion without iontophoresis (FIGS. 8-11 and FIGS. 16A and B). A comparison between the left cheek and left periorbit is shown in FIG. 12 and FIG. 13. A comparison between the left cheek and left perioral is shown in FIG. 14 and FIG. 15. The overall subjective and objective half-face scores for hyaluronic acid-wetted versus saline-wetted microdermabrasion with iontophoresis are shown in FIG. 17-FIG. 20.

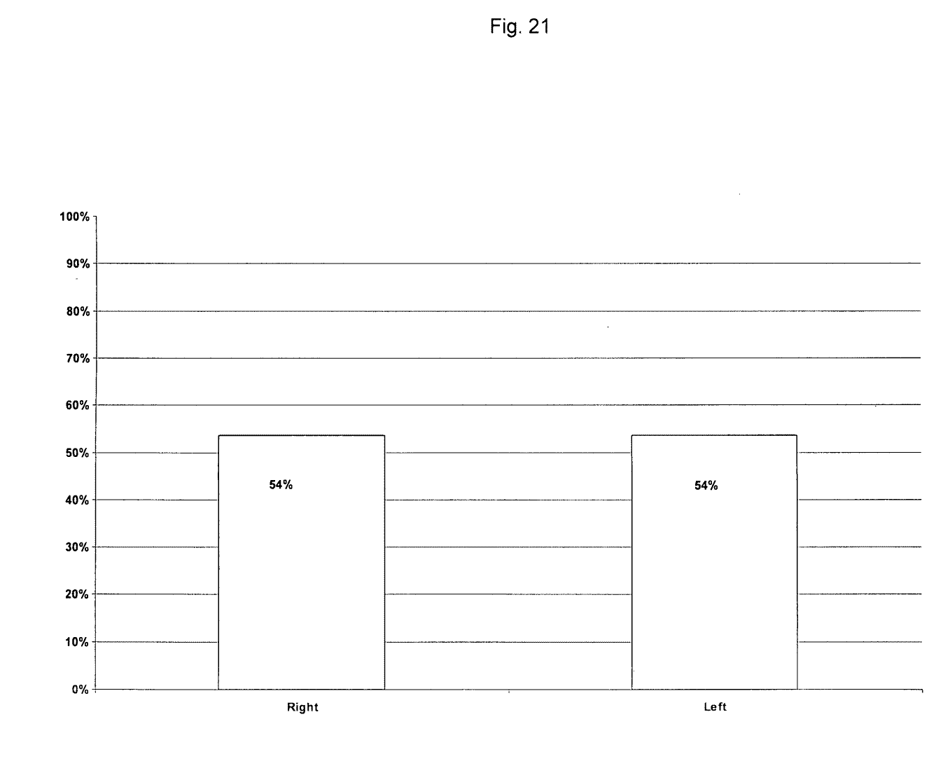

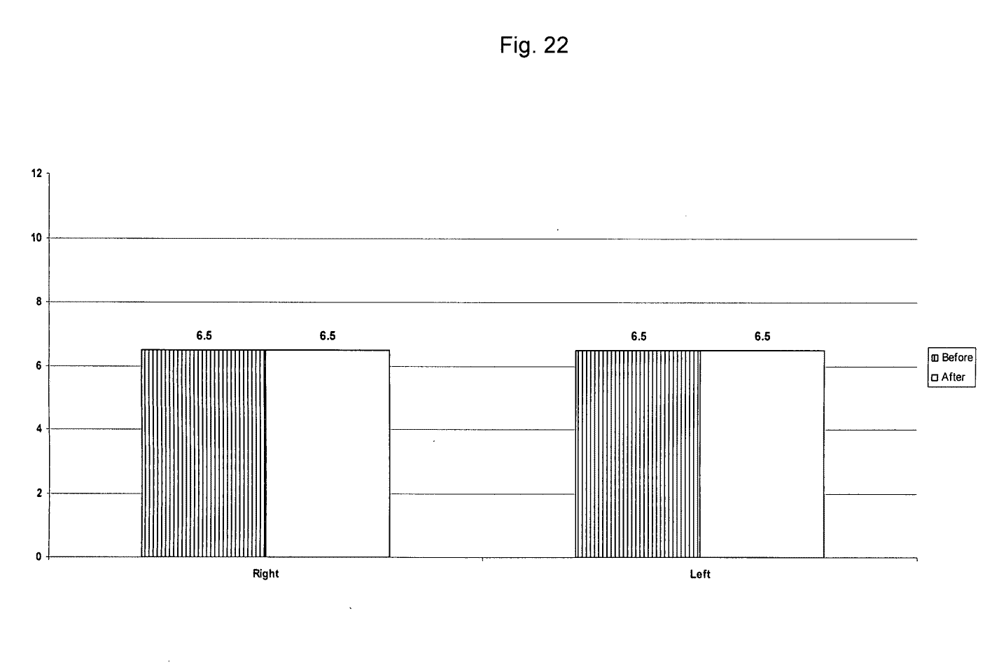

The gains that were seen with 30% hyaluronic acid-wetted microdermabrasion without iontophoresis were equal to the gains seen with iontophoresis. These gains were significant both by objective panel photographic assessment as well as by patient subjective assessment, with gains approaching 80 to 100% over baseline assessments. Additional gains could not be demonstrated with the use of iontophoresis over and above the rather dramatic gains that were observed with the use of 30% hyaluronic acid-wetted particle free microdermabrasion alone (FIG. 21 and FIG. 22). However, this would not exclude additional enhancement of benefit in modifications of iontophoresis application. Hyaluronic acid-wetted particle-free microdermabrasion holds great promise both as an adjunctive as well as a primary treatment in the treatment of facial aging.

In view of the many possible embodiments to which the principles of our disclosure can be applied, it should be recognized that the illustrated embodiment is only a preferred example of the disclosure and should not be taken as a limitation on the scope of the disclosure. Rather, the scope of the disclosure is defined by the following claims.

We claim:

1. A method for improving skin quality, comprising:
applying a composition comprising a therapeutically effective concentration of Botulinum toxin (BTX) to a skin surface with concurrent particle-free microdermabrasion treatment; wherein the application of the composition with concurrent particle-free microdermabrasion treatment improves skin quality.

2. The method of claim 1, wherein applying the composition comprising a therapeutically effective concentration of BTX comprises applying the composition comprising about 20 to 200 units equivalents of BTX.

3. The method of claim 1, further comprising applying at least about 20-30% weight per volume (W/V) of a pre-wetting agent, wherein the pre-wetting agent is selected from the group consisting of hyaluronic acid, chondroitin sulfate, and collagen.

4. The method of claim 3, wherein the pre-wetting agent is about a 25% W/V.

5. The method of claim 3, wherein the pre-wetting agent is about a 30% W/V.

6. The method of claim 3, wherein the pre-wetting agent comprises hyaluronic acid.

7. The method of claim 3, wherein the BTX and pre-wetting agent are administered in a single composition.

8. The method of claim 1, wherein the particle-free microdermabrasion treatment is performed using a wand containing an abrasive coated tip.

9. The method of claim 1, wherein the improvement is at least 50% as compared to prior to treatment.

10. The method of claim 1, wherein the improvement is at least 80% as compared to prior to treatment.

11. The method of claim 1, wherein the improvement is at least 100% as compared to prior to treatment.

12. The method of claim 1, wherein the method is performed more than once on a single subject.

13. The method of claim 12, wherein the method is performed twice within a two-week period on a single subject.

14. The method of claim 12, wherein the method is performed weekly on a single subject.

15. The method of claim 12, wherein the method is performed monthly on a single subject.

16. The method of claim 1, wherein the skin is periorbit, lip, cheek, nasolabial fold, upper lip rhytides, forehead, neck, back, hands or shoulder skin or a combination thereof.

17. The method of claim 1, wherein the skin quality improvement comprises a reduction in roughness, dryness, or scaliness, a reduction in fine lines and wrinkles, a reduction in scarring, an alteration in texture, or a reduction in age spots.

18. The method of claim 1, further comprising treating the skin surface with iontophoresis treatment.

19. A composition for improving skin quality, comprising a pre-wetting composition comprising at least about 20-30% weight/volume (W/V) of a pre-wetting agent, and a composition comprising Botulinum toxin comprising 20 to 200 units of Botulinum toxin, wherein the pre-wetting agent is selected from the group consisting of hyaluronic acid, chondroitin sulfate, and collagen.

20. The composition of claim 19, further comprising a preservative, an antimicrobial substance, a vitamin, a plant extract, a conditioning agent, or any combination thereof.

21. A kit for improving skin quality, comprising:
(a) a first composition comprising at least about 20%-30% of a pre-wetting agent, wherein the pre-wetting agent is selected from the group consisting of hyaluronic acid, chondroitin sulfate, and collagen; and a second composition comprising Botulinum toxin; or
(b) a single composition comprising a pre-wetting composition comprising at least about 20-30% of a pre-wetting agent, wherein the pre-wetting agent is selected from the group consisting of hyaluronic acid, chondroitin sulfate, and collagen; and a Botulinum toxin;
(c) and instructions for use.

22. The kit of claim 21, wherein the kit comprises a single composition comprising the pre-wetting agent comprising at least about 20-30% hyaluronic acid and Botulinum toxin, and instructions for use.

23. The kit of claim 21, wherein the kit comprises the first composition comprising at least about 20%-30% of a pre-wetting agent comprising at least about 20-30% hyaluronic acid and the second composition comprising Botulinum toxin, and instructions for use.

24. The kit of claim 21, further comprising an applicator.
